(19) United States
(12) Patent Application Publication
(10) Pub. No.: US 2025/0218600 A1
(43) Pub. Date: Jul. 3, 2025

(54) MINIMALLY INVASIVE GLUCOSE FORECASTING SYSTEMS, DEVICES, AND METHODS

(71) Applicant: SynchNeuro, Inc., Philadelphia, PA (US)

(72) Inventor: Casey HALPERN, Philadelphia, PA (US)

(21) Appl. No.: 18/850,022

(22) PCT Filed: Mar. 21, 2023
(86) PCT No.: PCT/US2023/064760

§ 371 Date: Sep. 23, 2024

Related U.S. Application Data
(60) Provisional application No. 63/322,086, filed on Mar. 21, 2022.

(51) Int. Cl. G16H 50/50; A61B 5/00; A61B 5/369; G16H 40/67

(52) U.S. Cl. CPC G16H 50/50; A61B 5/369; A61B 5/6814; A61B 5/742; G16H 40/67

(57) ABSTRACT

Glucose forecasting systems and methods that include a minimally invasive scalp-worn behind-the-ear EEG device that includes first and second sensors. With an application on a personal device, analyzing the processed EEG signals with a trained forecasting model and forecasting future glucose levels, of the subject; causing the personal device to visually present on a display information that is indicative of the forecasted future glucose levels.

### CROSS REFERENCE TO RELATED APPLICATIONS

This application claims priority to U.S. Provisional Application 63/322,086, filed Mar. 21, 2022 the entire disclosure of which is incorporated by reference herein for all purposes.

### INCORPORATION BY REFERENCE

All publications and patent applications mentioned in this specification are herein incorporated by reference to the same extent as if each individual publication or patent application was specifically and individually indicated to be incorporated by reference. PCT publication WO/2023/034820A1 is incorporated by reference herein in its entirety for all purposes. The entire disclosure of U.S. Prov. App. No. 63/238,583, filed Aug. 30, 2021, to which the WO/2023/034820A1 application claims priority, is also incorporated by reference herein in its entirety for all purposes.

### BACKGROUND OF THE DISCLOSURE

Currently, insulin pumps used for diabetes management generally achieve a peripheral glucose target range of 70 to 180 mg/dL<70% of the time, which is not ideal and still quite broad of a glycemic range. Moreover, hypoglycemic events remain disabling, suggesting there is much unmet need in optimizing this therapy for diabetes. System and methods are needed that can non-invasively, and from one or more discrete scalp regions, forecast future glucose levels.

### SUMMARY OF THE DISCLOSURE

One aspect of the disclosure is a method of forecasting future glucose levels of a subject.

In this aspect, the method may include sensing EEG signals from a subject with a behind-the-ear EEG device.

In this aspect, the method may include processing the sensed EEG signals.

In this aspect, the method may include, with an application on a personal device, analyzing processed EEG signals with a trained forecasting model and forecasting future glucose levels of the subject.

In this aspect, the method may optionally include, causing a personal device to visually present on a display information that is indicative of forecasted future glucose levels.

In this aspect, information that is indicative of forecasted future glucose levels may include a future period of time and forecasted glucose levels during the future period of time.

In this aspect, the method may further comprise communicating sensed EEG signals to a personal device.

One aspect of the disclosure is a computer executable method stored in a non-transitory memory of a personal device.

In this aspect, the method may include receiving sensed EEG data from a subject or information indicative of sensed EEG data from a subject.

In this aspect, the method may include analyzing the processed EEG signals with a trained forecasting model and forecasting future glucose levels of the subject.

In this aspect, the method may optionally include causing the personal device to visually present on a display information that is indicative of the forecasted future glucose levels.

One aspect of the disclosure is a glucose forecasting system (GFS).

In this aspect, the system may include a minimally invasive EEG device that includes first and second sensors, the EEG device sized, configured and adapted to be worn behind the ear of a subject and to sense EEG signals with the first and second sensors.

In this aspect, the system may include a personal device adapted to be in communication with the EEG device. The personal device may be further adapted to receive and process sensed EEG signals or information indicative of sensed EEG signals, and analyze the processed EEG signals with a trained forecasting model to forecast future glucose levels of the subject. In this aspect, a personal device is optionally further adapted to visually present on a display of the personal device information that is indicative of the forecasted future glucose levels.

### BRIEF DESCRIPTION OF THE DRAWINGS

FIG. 1 illustrates a system including an intracranial device.

FIG. 2 is a block diagram for exemplary controllers herein.

FIG. 3 illustrates an exemplary flow chart for an exemplary process for predictively managing glucose levels.

FIG. 4 is an exemplary flow chart of an exemplary process for predicting glucose levels based on brain activity data.

FIG. 5 illustrates an exemplary glucose forecasting system (“GFS”) including a plurality of components.

FIG. 6 is a non-limiting example of a visual representation of forecasted blood glucose levels on a display of a device (optionally a personal device), such as a smartphone or other computing device.

### DETAILED DESCRIPTION

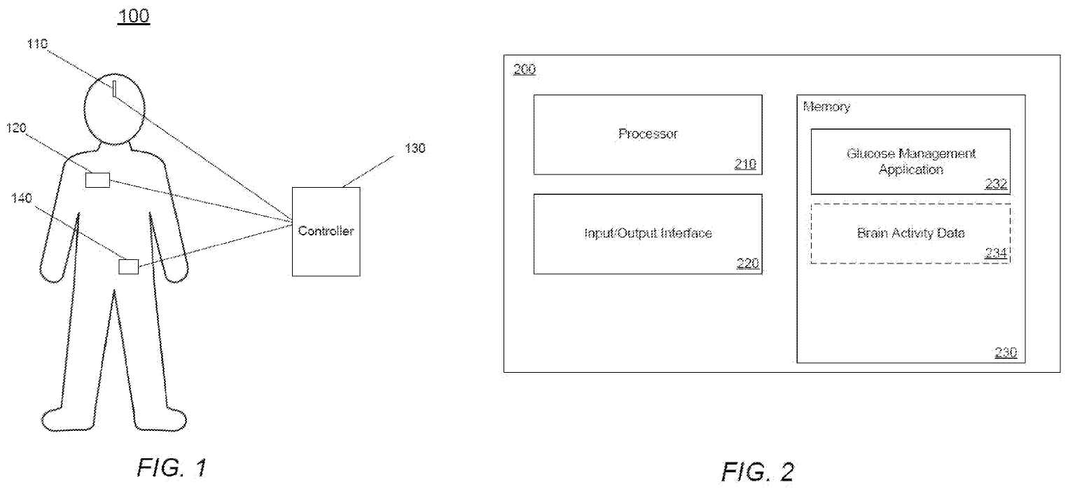

The disclosure is related to glucose forecasting systems and methods of use that include a behind-the-ear (or otherwise in close proximity to the ear), scalp-worn EEG device (EEG sensor). Much of the disclosure of PCT publication WO/2023/034820A1 (published Mar. 9, 2023) is expressly incorporated by reference below in paragraphs [0025]-[0041] and in FIGS. 1-4 herein, which may (but not necessarily) provide exemplary support and basis for one or more aspects of the glucose forecasting system and methods of use herein that include a minimally invasive behind-the-ear scalp-worn EEG sensor. The entire disclosure of U.S. Prov. App. No. 63/238,583, filed Aug. 30, 2021, to which the WO/2023/034820A1 application claims priority, is also incorporated by reference herein in its entirety for all purposes.

Metabolic syndromes and diabetes are increasingly prevalent health conditions, now affecting a broader range of ages in our global population. Specifically, the significant morbidity and mortality associated with diabetes have created a major toll on the healthcare system, including extensive costs, both personal and societal, in the form of medical expenditures for the disease itself and loss of workforce productivity from disability associated with disease progression. Hence, the ability to prevent development of diabetes and its subsequent complications is a high-impact area of public health for intervention. Additionally, eating behavior and physiologic regulation of the body's metabolic and weight balance entails a complex interplay of hormonal signaling and behaviors. In this context, close monitoring and control of blood glucose levels has been shown to be one of the best and most reliable methods to prevent complications of both hypo- and hyperglycemia.

Controlling blood glucose is important beyond the scope of diabetes, as both hyper- and hypoglycemia in hospitalized and critically-ill patients are associated with increased cost, length of stay, morbidity, and morality. Patients, especially those in intensive care units may suffer from stress-related hyperglycemia as a result of severe injuries and illnesses, e.g. traumatic brain injury (TBI), intracranial hemorrhage, stroke, subarachnoid hemorrhage (SAH), and many others. Conservative glycemic control has been associated with better outcomes in these patients.

Current iterations of continuous glucose monitors (CGMs) rely on interstitial glucose measurements as a proxy for blood glucose levels, which have an intrinsic lag time and are subject to interference by medications and extreme blood glucose values. Conventional CGMs are also unable to anticipate abnormal glucose levels; as a reactive modality, they can only respond to hypo or hyperglycemia once the abnormality has already occurred. In addition, patients with severe disease may have chronically elevated glucose levels that are refractory to conventional treatment options including continuous insulin infusion. Systems and methods described herein seek to rectify these limitations by predicting what a patient's glucose levels will be in the next several hours. Using this information, preemptive treatment can be delivered to the patient in order to avoid deleterious glucose levels from occurring.

In many embodiments, predictive glucose management (PGM) systems and methods described with respect to FIGS. 1-4 decode brain activity in order to predict the patient's future glucose levels. In a variety of embodiments, brain activity is measured using a non-invasive modality such as electroencephalography (EEG), functional near-infrared spectroscopy (fNIRS), magnetoencephalography (MEG), or any other modality as appropriate to the requirements of specific applications of embodiments herein. However, brain activity can be recorded using intracranial sensors if available, such as (but not limited to) deep brain stimulation (DBS) systems, or ECoG. In various embodiments, PGM systems are wearable or otherwise minimally invasive with respect to a patient's life outside of a clinical setting.

In numerous embodiments, systems and methods described herein involve closed-loop management of glucose levels, where preemptive treatment is provided to the patient to avoid hyper- or hypo-glycemia. For example, patients may be delivered long-acting insulin, an insulin analogue, and/or any other hyperglycemia control drug as appropriate to the requirements of specific applications in anticipation of future glucose changes. By way of further example, brain stimulation may be provided in order to perturb the glucose encoding network in the brain in order to alter blood glucose levels for the subsequent several hours. Brain stimulation may be provided by an already implanted DBS electrode depending on implantation location, any other type of implanted electrode, or via a non-invasive brain stimulation modality such as (but not limited to) transcranial magnetic stimulation (TMS), transcranial direct current stimulation (tDCS), transcranial focused ultrasound (tFUS), and/or any other modality as appropriate to the requirements of specific applications. In various embodiments, brain stimulation modalities may be utilized as an adjunct to insulin when the patient is refractory to standard treatments. PGM system architectures are discussed in further detail below.

PGM systems record and decode brain activity to estimate likely glucose levels of a patient in the next several hours. Typically, the predictions are accurate out for at least 2-8 hours, although depending on the patient and condition, this number may increase. In many embodiments, PGM systems provide these predictions to the patient and/or medical professionals. However, in various embodiments, PGM systems are further capable of closed-loop glucose level control by continuously predicting future glucose levels and altering treatment (e.g. drug delivery rate, brain stimulation, etc.) to avoid the predicted deleterious change in glucose level. In this way, PGMs can perform as an artificial pancreas system with superior glucose management capabilities. In some embodiments, patient and/or medical professional authorization is required before treatment is delivered and/or modified by the PGM.

FIG. 1 illustrates an example PGM system architecture in accordance with an embodiment. PGM system 100 includes a brain activity recorder 110. In the illustrated embodiment, brain activity recorder 110 is a deep brain stimulation system. However, as can readily be appreciated, any brain activity recorder can be used, including those that are non-invasive as discussed above. In some embodiments, the brain activity recorder is a wearable device, rather than an implanted device. PGM system 100 further includes a CGM 120. In many embodiments CGMs are used to continuously confirm the accuracy of interstitial blood glucose predictions, and may further act as a redundant warning modality. However, CGMs may not be present in all PGM systems as appropriate to the requirements of specific applications.

A controller 130 is communicatively coupled with the brain activity recorder 110, the CGM 120, and an insulin infusion pump 140. In many embodiments, the communication between different components may not be direct. For example, a brain activity recorder may provide data to a CGM which in turn is provided to the controller rather than communicating with the controller directly. Indeed, as one of ordinary skill in the art would appreciate, any communications architecture can be used without departing from the scope or spirit of the disclosure herein.

In numerous embodiments, controllers process recorded brain activity to generate predictions regarding the patient's glucose levels. Controllers may provide predictions for only one of interstitial or blood glucose levels. In some embodiments both interstitial and blood glucose level predictions are computed. Further, controllers may be implemented using any of a variety of computing platforms. In various embodiments, the controller is a smart phone, a smart watch, a tablet computer, a personal computer, and/or any other personal wearable device. In some embodiments, the controller may be integrated into a medical device or a medical server system, e.g. a hospital computer network, or cloud medical system.

In various embodiments, insulin infusion pumps can variably infuse insulin as dictated by the controller. Further, other drugs rather than insulin may be provided via a similar infusion pump depending on the needs of the patient. As can be readily appreciated, many PGM systems may not include any infusion pumps if drug delivery is unadvisable for the particular patient. Similarly, PGMs may further include methods for delivering brain stimulation as an alternative treatment. In various embodiments, the brain activity recorder may also function as a brain stimulation device. Indeed, any number of different PGM system architectures can be used depending on the needs of a specific patient as appropriate to the requirements of specific applications.

Turning now to FIG. 2, a block diagram for any of the controllers herein is illustrated. Controller 200 includes a processor 210. In many embodiments, the processor is a logic circuit capable of executing instructions such as (but not limited to) a central processing unit (CPU), a graphics processing unit (GPU), a field programmable gate array (FPGA), an application-specific integrated circuit (ASIC), and/or any combination thereof. In many embodiments, more than one processor can be used. Controller 200 further includes an input/output (I/O) interface 220. The I/O interface can be used to communicate with different PGM system components and/or 3^{rd} party components such as (but not limited to) displays, speakers, CGMs, brain activity recorders, stimulation devices, infusion pumps, cellphones, medical devices, computers, and/or any other component via wired or wireless connections.

Controller 200 further includes a memory 230. The memory 230 can be made of volatile memory, nonvolatile memory, or any combination thereof. The memory 230 contains a glucose management application 232. The glucose management application can direct the processor to carry out various PGM processes as described herein. In many embodiments, the memory 230 further contains brain activity data obtained from brain activity recorders. Brain activity data can describe brain activity as a signal or set of signals. In some embodiments, brain activity data includes waveforms recorded by sensor electrodes. In various embodiments, one or more waveforms are recorded for each electrode (“channel”). In a number of embodiments, the brain activity data describes the spectral profile of broadband brain activity. In a variety of embodiments, the glucose management application configures the processor to act as a multivariate decoder for brain activity data. As can be readily appreciated, controllers can be manufactured in different ways using similar computing components without departing from the scope or spirit of the disclosure. PGM processes are discussed in further detail below.

Predictive Glucose Management. PGM processes involve the collection and use of brain activity to predict future glucose levels of a patient. In numerous embodiments, treatment recommendations or the treatments themselves can be triggered by a prediction of hyper- or hypoglycemia in order to stabilize the glucose levels at a healthier range. Peripheral glucose levels tend to largely follow circadian dynamics and are strongly coherent to intracranial high frequency activity (HFA, 70-170 Hz) across multiple brain regions. As such, whole brain activity can be used in the predictive modeling process. In some embodiments, brain activity data from known glucose-sensors such as the hypothalamus, amygdala, and hippocampus are used instead of or in conjunction with brain activity from other regions and/or the whole brain.

In order to process data coming from one or more brain activity recorders, a machine learning model can be trained. In some embodiments, the training process is performed using data acquired from the patient on which the trained model will be used. In various embodiments, the model can be pre-trained on standardized data and training can be completed using patient data. In various embodiments, the model is continuously refined using predictions and subsequent validation as measured using a CGM. While linear models are often considered to be less predictive than more modem machine learning models, in many embodiments a linear model is sufficient for accurate prediction. However, in various embodiments, more complex predictive machine learning models can be used, such as (but not limited to) other types of regression models, neural networks, and others as appropriate to the requirements of specific applications.

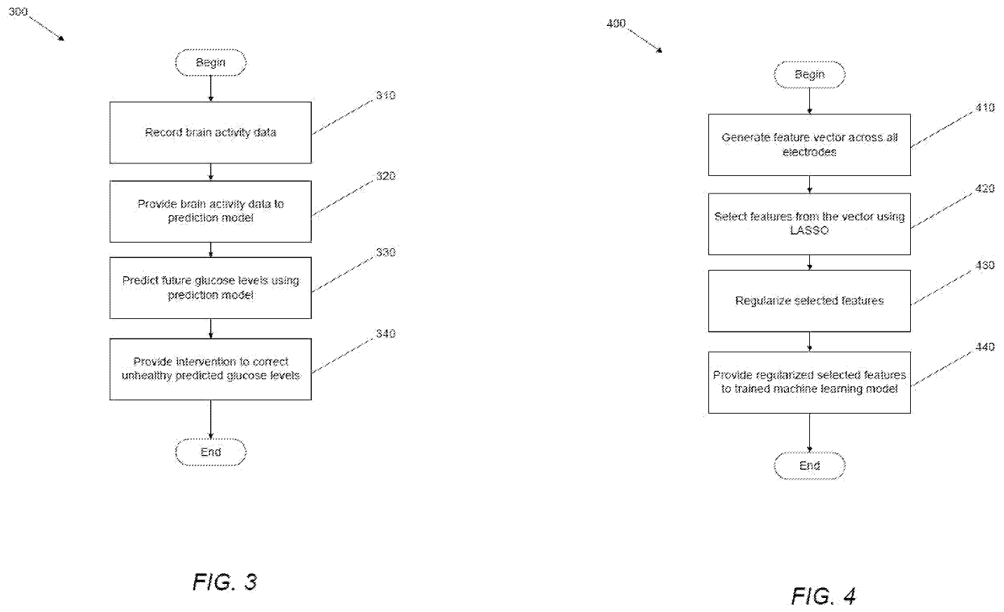

FIG. 3 illustrates an exemplary flow chart for an exemplary PGM process for predictively managing glucose levels. Process 300 includes recording (310) brain activity using a brain activity recorder, and providing (320) the brain activity data to a trained prediction model. The prediction model predicts (330) future glucose levels. In many embodiments, the certainty of the prediction may decrease the further in the future the prediction is made. In various embodiments, multiple predictions are provided at different time points, and only those above a predetermined confidence threshold as determined by a medical professional are used. In some embodiments, a hard limit is set on how far ahead the predictions are made for. If a hypo- and/or hyperglycemic state is predicted, a medical intervention is provided (340) to avoid the unhealthy dip or spike, respectively, in glucose levels. In various embodiments, the medical intervention is automatically provided, e.g. via control of an infusion pump and/or via brain stimulation. In various embodiments, a warning is provided to the patient and/or medical professional that an unhealthy glucose level is predicted. In some embodiments, confirmation is required before the medical intervention is provided.

While a particular process is illustrated in FIG. 3, as can readily be appreciated, various modifications can be made without departing from the scope or spirit of the disclosure. For example, pre-recorded brain activity data can be provided and used to make predictions. Further, interventions need not be recommended nor provided in all cases. In many situations, it is beneficial to merely have a warning.

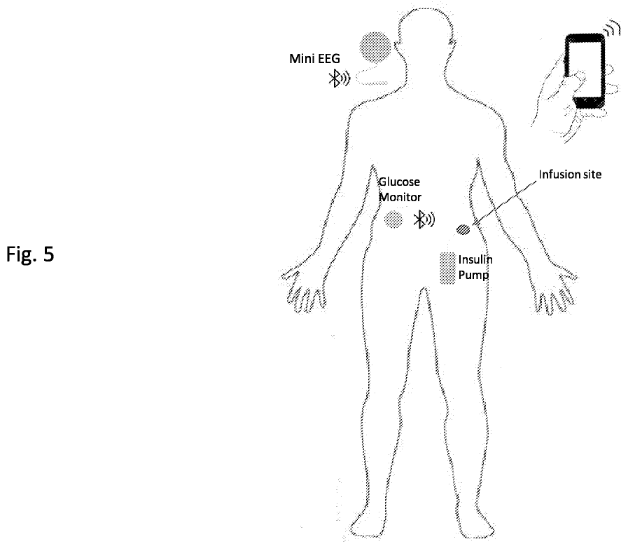

FIG. 4 is an exemplary flow chart of an exemplary PGM process for predicting glucose levels based on brain activity data. Process 400 includes generating (410) a feature vector across all electrode (or sensor) channels. In numerous embodiments, all frequency bands across all channels are flattened into a single feature vector. A Least absolute shrinkage and selection operator (LASSO) model is used to select (420) a subset of features from the feature vector for regularization (430). The regularized features are provided (440) to the trained machine learning model to produce one or more predictions. In numerous embodiments, a similar process is used to train the model using labeled training data from the patient and/or other patients. While specific machine learning models are discussed herein, many different machine learning models can be used without departing from the scope or spirit of the invention.

The disclosure below, including exemplary FIG. 5, describes systems and methods adapted to forecast future glucose levels non-invasively using a behind-the-ear scalp EEG sensor location. The entire disclosure of U.S. Pat. No. 11,020,035 is incorporated by reference herein for all purposes with respect to an exemplary wearable EEG sensor, any aspect of which may be incorporated into the EEG device (sensor) that is illustrated in FIG. 5.

FIG. 5 illustrates an exemplary glucose forecasting system (“GFS”) including a plurality of components. The GFS includes a wearable EEG sensor (labeled “Mini EEG,”) which may include first and second paired electrodes. The EEG is adapted, sized and configured to be worn behind the ear and to measure EEG signals from the scalp. The GFS system may also include an interstitial glucose-sensing insulin pump (e.g., a glucose monitor (such as a continuous glucose monitor) plus an insulin pump with infusion site) worn on the stomach. The GFS may also include an “App” stored on a smartphone (or smart wearable device) that is adapted and configure to receive and analyze the EEG and glucose data continuously (and optionally as well as the delivered insulin) to produce a “forecast,” which may optionally be used so the insulin pump can deliver insulin. The App may optionally communicate with an online, secure patient management portal from which physicians remotely in clinic and patients at home or in clinic can see trends in the insulin delivery and EEG signal patterns, as well as their relationship.

The GFSs herein can be adapted and configured to forecast glucose levels and variations up to 6 hours prior to the actual change in level. Once a shift or change in glucose is forecasted, the App can optionally communicate a command to the insulin pump to deliver the adequate amount of needed insulin before extremes in glucose levels occur. The GFSs herein may optionally incorporate a glucose monitor (i.e., GFSs may not include a glucose monitor) until EEG-guided insulin titrations are further validated for the individual. GFSs may also rely on or utilize additional standard monitoring techniques, such as finger prick blood glucose monitoring. An exemplary benefit of GFSs over standard pumps is the ability to treat trends hours before they approach risky levels (either high or low glucose levels). Indeed, hypoglycemia is the most common problem seen with insulin-based therapies today. The GFSs herein provide for more safely titrating up and down predictive algorithms. Moreover, an optional closed-loop approach prevents dangerous out of range glucose levels, and may optionally even avoid having to monitor their own glucose levels. The GFSs herein may also be adapted to inform best dosing of long-acting insulin injections.

The GFSs herein may optionally be configured to provide information related to or about the glucose forecast. For example only, the App may be adapted to display at least a portion of the glucose forecast on a screen of the device (e.g., phone, wearable), and/or a predicted time or time period during which the forecast glucose levels will drop below a threshold or rise about a threshold.

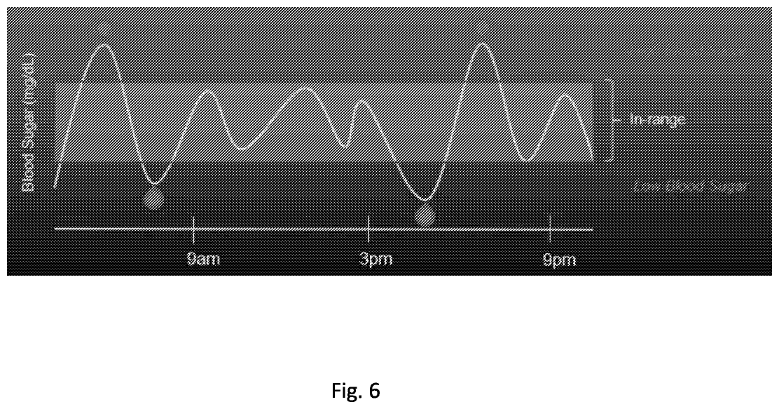

FIG. 6 is a non-limiting example of a visual representation of forecasted blood glucose levels on a display of a device, such as a smartphone or other computing device. For example, an App may comprise a computer executable method that is adapted to present forecasted future blood glucose levels, the computer executable method stored in a non-transitory memory, the method comprising: receiving as input extracranially-sensed EEG data or information indicative of extracranially-sensed EEG from a subject (from a behind the ear wearable EEG sensor); and causing a visual representation of the forecasted future blood glucose levels to be displayed on a display of a device. In exemplary FIG. 6, time is on the X-axis and the blood sugar levels on the Y-axis. The left side of the X-axis can be considered 3 am, where predicted levels that are out of range are shown. Visual indicators (e.g., the red icons) can indicate predicted peaks and valleys. The range may be user-adjustable on the display so the user can change the high or low levels for the range that is shown as “in-range.”

As mentioned above, existing closed-loop systems for detecting glucose levels and automatically delivering insulin are operating in real-time. They are reacting to peaks and dips in glucose as those changes happen. The GSFs described herein are adapted to sense EEG with a behind the ear wearable EEG sensor and forecast shifts in glucose levels hours before they occur, as well as optionally titrate the insulin based on this forecast before extremes occur in the glucose levels. Additionally, titrating insulin over time can dramatically decrease the risk of overcorrecting and causing symptomatic hypoglycemia, the most common disabling side effect of all current insulin-based treatments.

Even if not specifically indicated, one or more methods or techniques described in this disclosure (e.g. any of the computer executable methods) may be implemented, at least in part, in hardware, software, firmware or any combination thereof. For example, various aspects of the techniques or components may be implemented within one or more processors, including one or more microprocessors, digital signal processors (DSPs), application specific integrated circuits (ASICs), field programmable gate arrays (FPGAs), programmable logic circuitry, or the like, either alone or in any suitable combination. The term “processor” or “processing circuitry” may generally refer to any of the foregoing circuitry, alone or in combination with other circuitry, or any other equivalent circuitry.

Such hardware, software, or firmware may be implemented within one device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read only memory (ROM), non-volatile RAM (NVRAM), electrically erasable programmable ROM (EEPROM), Flash memory, and the like. The instructions may be executed by a processor to support one or more aspects of the functionality described in this disclosure.

1. A method of forecasting future glucose levels of a subject, comprising:
sensing EEG signals from a subject with a behind-the-ear EEG device;
processing the sensed EEG signals;
with an application on a personal device, analyzing the processed EEG signals with a trained forecasting model and forecasting future glucose levels of the subject; and
causing the personal device to visually present on a display information that is indicative of the forecasted future glucose levels.

2. The method of claim 1, wherein the information that is indicative of the forecasted future glucose levels includes a future period of time and forecasted glucose levels during the future period of time.

3. The method of claim 2, wherein the information that is indicative of the forecasted future glucose levels comprises a graph with time on a first axis and forecasted glucose levels on a second axis.

4. The method of claim 1, further comprising communicating the sensed EEG signals to the personal device.

5. A method of forecasting future glucose levels of a subject, comprising
sensing EEG signals from a subject with a behind-the-ear EEG device;
processing the sensed EEG signals; and
with an application on a personal device, analyzing the processed EEG signals with a trained prediction model and forecasting future glucose levels of the subject.

6. The method of claim 5, further comprising communicating the sensed EEG signals to the personal device.

7. A computer executable method stored in a non-transitory memory of a personal device, comprising:
receiving sensed EEG data from a subject or information indicative of sensed EEG data from a subject;
analyzing the processed EEG signals with a trained forecasting model and forecasting future glucose levels of the subject; and
causing the personal device to visually present on a display information that is indicative of the forecasted future glucose levels.

8. The computer executable method of claim 7, wherein the causing step comprises causing the personal device to visually present on the display a future period of time and forecasted glucose levels during the future period of time.

9. The computer executable method of claim 8, wherein the causing step comprises causing the personal device to visually present on the display a graph with time on a first axis and forecasted glucose levels on a second axis.

10. A glucose forecasting system (GFS), comprising:
a minimally invasive EEG device that includes first and second sensors, the EEG device sized, configured and adapted to be worn behind the ear of a subject and to sense EEG signals with the first and second sensors; and
a personal device adapted to be in communication with the EEG device, the personal device further adapted to,
receive and process the sensed EEG signals or information indicative of the sensed EEG signals, and
analyze the processed EEG signals with a trained forecasting model to forecast future glucose levels of the subject.

11. The glucose forecasting system of claim 10, wherein the personal device is further adapted to visually present on a display of the personal device information that is indicative of the forecasted future glucose levels.
